(19) United States
(12) Patent Application Publication
(10) Pub. No.: US 2026/0062364 A1
(43) Pub. Date: Mar. 5, 2026

(54) METHANE PURIFICATION APPARATUS

(71) Applicant: ISUZU MOTORS LIMITED, Yokohama-shi (JP)

(72) Inventors: Ryusuke FUJINO, Fujisawa-shi (JP); Teppei OHORI, Fujisawa-shi (JP); Hisashi OZAWA, Fujisawa-shi (JP)

(21) Appl. No.: 19/298,936

(22) Filed: Aug. 13, 2025

(30) Foreign Application Priority Data
Sep. 4, 2024 (JP) 2024-152456

(51) Int. Cl. C07C 7/148; B01J 8/02

(52) U.S. Cl. CPC C07C 7/14816; B01J 8/0285; B01J 2208/00398

(57) ABSTRACT

A methane purification apparatus includes a flow path through which a gas containing methane flows, an ozone supply unit that supplies ozone to the gas, and a non-metallic heater that is provided downstream of the ozone supply unit in the flow path and heats the gas and the ozone. The heater supports a catalyst for purifying the methane by causing the ozone to react with the methane.

### CROSS-REFERENCE TO RELATED APPLICATIONS

The present application claims priority to Japanese Patent Applications number 2024-152456, filed on September 4, 2024 contents of which are incorporated herein by reference in their entirety.

### BACKGROUND OF THE INVENTION

The present disclosure relates to a methane purification apparatus for purifying methane in a gas.

Japanese Unexamined Patent Application Publication No. 2021-505376 discloses a technique in which a gas containing methane and ozone is supplied to a catalyst, and the methane and ozone react on the catalyst to purify the methane. Specifically, the methane is purified by being decomposed into carbon dioxide and water through a reaction with ozone.

However, in the above technique, when the temperature of the catalyst is low, the reaction between methane and ozone on the catalyst is less likely to proceed, making it difficult to efficiently purify the methane.

### BRIEF SUMMARY OF THE INVENTION

The present disclosure focuses on this point, and an object thereof is to promote a reaction between methane and ozone on a catalyst.

An aspect of the present disclosure provides a methane purification apparatus including a flow path through which a gas containing methane flows, an ozone supply unit that supplies ozone to the gas, and a non-metallic heater that is provided downstream of the ozone supply unit in the flow path and heats the gas and the ozone, wherein the heater supports a catalyst for purifying the methane by causing the ozone to react with the methane.

### BRIEF DESCRIPTION OF THE DRAWINGS

FIG. 1 schematically shows a configuration of a methane purification apparatus 1 according to an embodiment.

FIG. 2 schematically shows an example of a configuration of a catalytic heater 32.

FIG. 3 schematically shows a first temperature control by a heater control unit 64.

FIG. 4 schematically shows a second temperature control by the heater control unit 64.

FIG. 5 is a schematic view illustrating a modification.

### DETAILED DESCRIPTION OF THE INVENTION

Hereinafter, the present disclosure will be described through exemplary embodiments of the present disclosure, but the following exemplary embodiments do not limit the disclosure according to the claims, and not all of the combinations of features described in the exemplary embodiments are necessarily essential to the solution means of the disclosure.

### <Configuration of methane purification device>

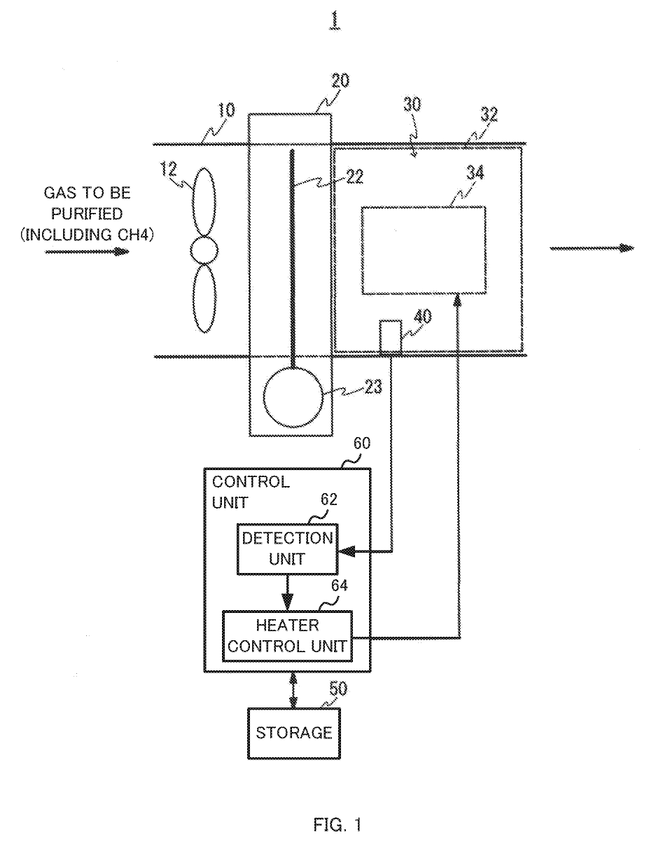

FIG. 1 schematically shows a configuration of a methane purification apparatus 1 according to an embodiment.

The methane purification apparatus 1 is an apparatus for purifying a gas to be purified, which is a gas containing methane. Here, the gas to be purified is air containing methane. The methane purification apparatus 1 can be installed in a factory, a house, or the like. The methane purification apparatus 1 includes a flow path 10, a fan 12, an ozone supply unit 20, a methane decomposition unit 30, a temperature sensor 40, a storage 50, and a control unit 60.

The flow path 10 forms a flow path through which the gas to be purified containing methane flows. The flow path 10 is a circular tube, for example. The fan 12, the ozone supply unit 20, the methane decomposition unit 30, and the temperature sensor 40 are provided in the flow path 10.

The fan 12, by rotating, draws the gas to be purified containing methane into the flow path 10. The gas to be purified drawn in by the fan 12 flows toward the methane decomposition unit 30 located downstream of the fan 12. The fan 12 is provided in the flow path 10, but the present disclosure is not limited thereto, and the fan 12 may be provided outside the flow path 10.

The ozone supply unit 20 is provided downstream of the fan 12 in the flow path 10, and supplies ozone to the gas to be purified drawn in by the fan 12. The ozone supply unit 20 generates ozone and supplies the ozone to the gas to be purified containing methane. The ozone flows toward the methane decomposition unit 30 together with the gas to be purified. Specifically, the ozone flows toward the methane decomposition unit 30 in a state of being mixed with the gas to be purified.

The ozone supply unit 20 generates ozone by performing a process of silent discharge on the gas to be purified (a so-called silent discharge method), for example. Specifically, the ozone supply unit 20 generates ozone by applying an AC voltage from a power source 23 to an electrode 22 covered with a dielectric material such as glass. However, the present disclosure is not limited to the above, and the ozone supply unit 20 may generate ozone by performing a process of electrolyzing water (a so-called electrolysis method), or by irradiating the gas to be purified with ultraviolet rays (a so-called ultraviolet lamp method).

The methane decomposition unit 30 is provided downstream of the ozone supply unit 20 in the flow path 10, and has a function of decomposing methane in the gas to be purified using ozone. The methane decomposition unit 30 includes a catalyst for decomposing methane, and decomposes methane into water and carbon dioxide by causing ozone and methane to react on the catalyst.

Methane is decomposed by reacting with ozone on the catalyst, but when the temperature of the catalyst is low, the reaction between methane and ozone does not proceed sufficiently, making it difficult to decompose the methane. This is because the catalyst exhibits a property of promoting the reaction between methane and ozone more effectively as the temperature increases.

Therefore, in the present embodiment, in order to promote the reaction between methane and ozone on the catalyst, the methane decomposition unit 30 includes a catalytic heater 32 that functions as a heater for heating the gas to be purified and ozone. The catalytic heater 32 supports the catalyst. In this case, the gas to be purified and ozone, heated by the catalytic heater 32, contact the catalyst, which increases the temperature of the catalyst and promotes the reaction between methane and ozone on the catalyst.

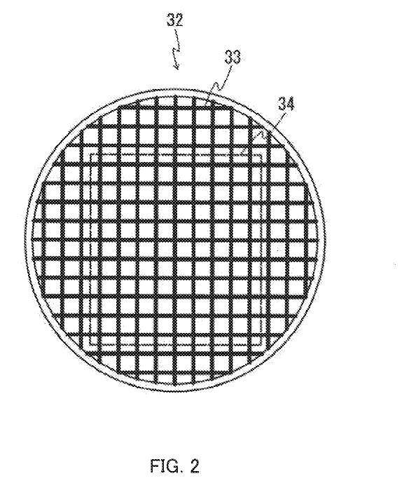

FIG. 2 schematically shows an example of a configuration of the catalytic heater 32. The catalytic heater 32 is located downstream of the ozone supply unit 20, and heats the gas to be purified that has passed through the ozone supply unit 20 and ozone generated by the ozone supply unit 20. As shown in FIG. 2, the catalytic heater 32 includes a carrier 33 having a honeycomb structure. The catalytic heater 32 includes a heat generating unit 34 that generates heat when electricity is supplied to the inside of the carrier 33, for example. Therefore, the carrier 33 of the present embodiment functions as a heating element.

The heat generating unit 34 includes an electric heating wire that converts electric energy into heat energy, for example. The heating wire exchanges heat with the gas to be purified and ozone to heat the gas to be purified and ozone and raise temperature of the gas to be purified and ozone. Here, the carrier 33 has a cylindrical shape as shown in FIG. 2, and has the heat generating unit 34 at a central portion thereof. However, the present disclosure is not limited thereto, and the carrier 33 may be a rectangular parallelepiped.

The catalytic heater 32 is made of a non-metal. Specifically, the carrier 33 of the catalytic heater 32 is made of silicon carbide. Since silicon carbide is electrically conductive, it generates heat when supplied with electricity. Accordingly, when the catalytic heater 32 is made of silicon carbide, the catalytic heater 32 can easily heat the gas to be purified and ozone in the flow path 10 appropriately.

Unlike the present embodiment, if the carrier 33 of the catalytic heater 32 is made of metal, ozone tends to react with the metal of the carrier 33, resulting in oxidation of the metal and conversion of the ozone into oxygen. In such a case, the amount of ozone available to react with methane decreases. Therefore, in the present embodiment, the catalytic heater 32 is made of a non-metallic material in order to prevent such a decrease in ozone.

Although a heating element made of ceramic can be used as the catalytic heater 32, a catalytic heater 32 made of silicon carbide can heat the gas to be purified and ozone more quickly than one made of ceramic. Furthermore, by forming a honeycomb structure using silicon carbide, a surface area of a heat generating portion of the catalytic heater 32 can be increased, allowing the gas to be purified and ozone to be heated more efficiently.

The catalytic heater 32 supports a catalyst for purifying methane by causing ozone to react with methane. Specifically, the carrier 33 of the catalytic heater 32 supports the catalyst. In other words, a catalyst layer to which the catalyst adheres is formed on the surface of the carrier 33. The catalyst of the catalyst layer includes any of a zeolite, an iron ion-exchanged zeolite, and a cobalt ion-exchanged zeolite. In the case of such a catalyst, ozone and methane easily react with each other even when the temperature of the catalyst is low.

Ozone and methane come into contact with the catalyst layer of the catalytic heater 32 and react on the catalyst layer. When the catalytic heater 32 heats the gas to be purified and ozone, the heated gas and ozone contact the catalyst layer, causing the temperature of the catalyst layer to rise. As a result, the reaction between ozone and methane on the catalyst layer is promoted.

It should be noted that, in the above description, the catalytic heater 32 (specifically, the carrier 33) is made of silicon carbide, but the present disclosure is not limited thereto. The catalytic heater 32 may be made primarily of barium titanate, for example, as long as it is made of a non-metallic material. Even in this case, the gas to be purified and ozone can be appropriately heated while preventing a decrease in ozone.

In addition, in the above description, the carrier 33 is assumed to have a honeycomb structure, but the present disclosure is not limited thereto. The carrier 33 may have a corrugated or mesh structure, as long as the catalyst can be supported on the surface of the carrier 33.

Returning to FIG. 1, the configuration of the methane purification apparatus 1 will be described.

The temperature sensor 40 is provided in the methane decomposition unit 30 and is a sensor for detecting temperature around the catalytic heater 32. Specifically, the temperature sensor 40 detects temperature of the gas to be purified and ozone flowing through the methane decomposition unit 30. The temperature sensor 40 is a thermistor or a thermocouple, for example.

The storage 50 includes a storage medium such as a Read Only Memory (ROM), a Random Access Memory (RAM), a Hard Disk Drive (HDD), or a Solid State Drive (SSD), for example. The storage 50 stores a program executed by the control unit 60 and various kinds of information for decomposing methane.

The control unit 60 includes a processor such as a Central Processing Unit (CPU). The control unit 60 causes the catalytic heater 32 to heat the gas to be purified and ozone by supplying electricity to the catalytic heater 32. As a result, methane and ozone react on the catalyst, whose temperature has been increased. The control unit 60 may be configured by a single processor, or may be configured by a plurality of processors or a combination of one or more processors and an electronic circuit. By executing the program stored in the storage 50, the control unit 60 functions as a detection unit 62 and a heater control unit 64. In the present embodiment, the heater control unit 64 corresponds to a temperature control unit.

The detection unit 62 detects temperature of the catalyst in the catalytic heater 32. The detection unit 62 detects the temperature of the catalyst in the catalytic heater 32, for example, by acquiring the temperature detected by the temperature sensor 40. For example, the detection unit 62 detects temperature detected by the temperature sensor 40 as the temperature of the catalyst. However, the present disclosure is not limited thereto, and the detection unit 62 may detect a value obtained by multiplying the temperature detected by the temperature sensor 40 by a predetermined coefficient as the temperature of the catalyst.

The heater control unit 64 controls the operation of the catalytic heater 32 that heats the gas to be purified and ozone. For example, when the ozone supply unit 20 starts supplying ozone to the gas to be purified, the heater control unit 64 operates the catalytic heater 32 to heat the gas to be purified and the ozone. The heater control unit 64 controls the operation of the catalytic heater 32 based on the temperature detected by the detection unit 62.

Ozone is known to undergo thermal decomposition when the temperature exceeds a predetermined first temperature (e.g., 150°C). When ozone is thermally decomposed, the amount of ozone available to react with methane decreases. Therefore, the heater control unit 64 operates the catalytic heater 32 so that the temperature of the catalyst is below the first temperature at which ozone decomposes. Specifically, the heater control unit 64 controls the supply of electricity to the catalytic heater 32 so that the temperature of the catalyst, as detected by the detection unit 62, is below the first temperature. Accordingly, thermal decomposition of ozone can be suppressed during operation of the catalytic heater 32.

When ozone reacts with methane on the catalyst, carbon dioxide and water are generated as described above, and the generated water may remain on the catalyst. If water remains on the catalyst, an area available for contact between the catalyst and ozone and methane is reduced, thereby inhibiting the reaction between ozone and methane.

Accordingly, in the present embodiment, to suppress the state in which water remains on the catalyst, the heater control unit 64 operates the catalytic heater 32 so that the temperature of the catalyst, as detected by the detection unit 62, is below the first temperature and at or above a second temperature (e.g., 100°C) at which moisture evaporates.

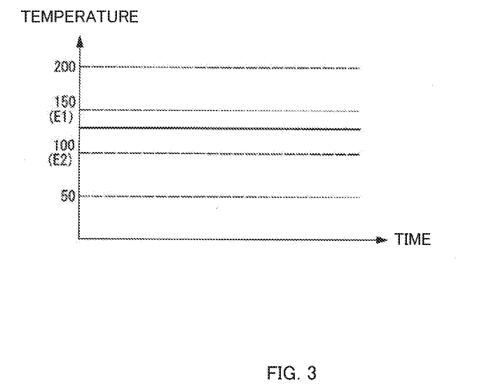

FIG. 3 schematically shows a first temperature control by the heater control unit 64. The heater control unit 64 operates the catalytic heater 32 so that the temperature of the catalyst is below a first temperature E1 (150°C) and at or above a second temperature E2 (100°C). Here, the heater control unit 64 operates the catalytic heater 32 so that the temperature of the catalyst is at about 130°C. In this case, a state in which moisture remains on the catalyst can be suppressed while suppressing thermal decomposition of ozone. As a result, the reaction between ozone and methane is promoted, thereby increasing the methane purification rate.

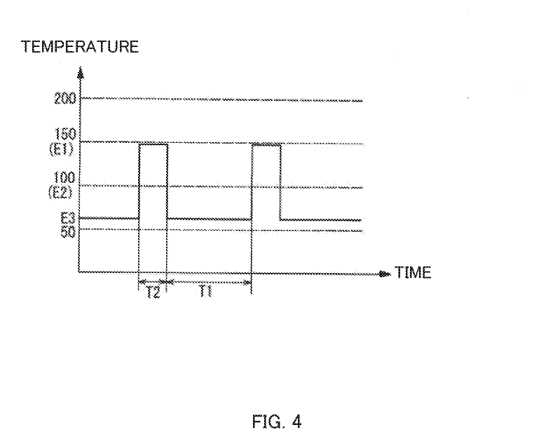

In the first temperature control described above, the heater control unit 64 operates the catalytic heater 32 so that the temperature of the catalyst is maintained constant, but the present disclosure is not limited thereto. For example, as illustrated in FIG. 4, the heater control unit 64 may alternately repeat a low-temperature control and a high-temperature control.

FIG. 4 schematically shows a second temperature control by the heater control unit 64. The heater control unit 64 performs the low-temperature control by operating the catalytic heater 32 so that the temperature of the catalyst, as detected by the detection unit 62, is maintained at a third temperature E3 (approximately 60°C), which is lower than the second temperature E2 (100°C), for a first time period T1. Further, the heater control unit 64 performs the high-temperature control by operating the catalytic heater 32 so that the temperature of the catalyst is maintained between the second temperature E2 and the first temperature E1 (150°C) for a second time period T2, which is shorter than the first time period T1. It should be noted that the second time period T2 is shorter than the first time period T1 (e.g., 10 minutes), and may be 1 minute, for example. In addition, the third temperature E3 is greater than 50°C and less than 100°C.

Then, the heater control unit 64 alternately repeats the low-temperature control and the high-temperature control. Specifically, the heater control unit 64 performs the high-temperature control intermittently between periods of the low-temperature control. When the catalyst in the catalytic heater 32 is a cobalt ion-exchanged zeolite, the second temperature control is performed because cobalt ion-exchanged zeolite exhibits a property of promoting the reaction between methane and ozone at the third temperature (low temperature). It should be noted that, when the first temperature control is performed, the catalyst may be an iron ion-exchanged zeolite, for example.

By performing the second temperature control, the reaction between methane and ozone can be promoted through the low-temperature control, while suppressing moisture from remaining on the catalyst through the high-temperature control.

When the catalyst is an iron ion-exchanged zeolite or a cobalt ion-exchanged zeolite, ozone may oxidize the metal component of the catalyst if the temperature of the catalyst exceeds 150°C, which may result in a decrease in ozone. In contrast, in the first temperature control and the second temperature control, the heater control unit 64 can suppress the decrease in ozone by setting the catalyst temperature to below 150°C (the first temperature).

### (Modification)

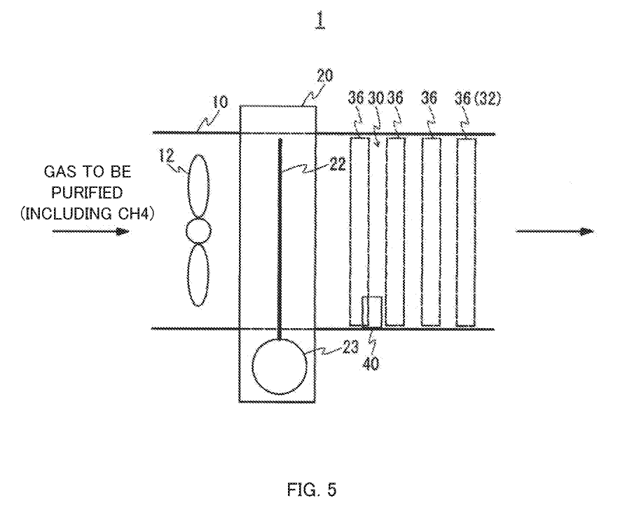

In the above description, the catalytic heater 32 is a structure having a honeycomb structure, but the present disclosure is not limited thereto. For example, the catalytic heater 32 may have a structure as shown in FIG. 5.

FIG. 5 is a schematic view illustrating a modification. In FIG. 5, only the configuration of the catalytic heater 32 differs from that shown in FIG. 1, and since the other components are the same as those in FIG. 1, detailed description thereof is omitted.

As shown in FIG. 5, the catalytic heater 32 according to the modification includes a plurality of flat plate-like heat generating plates 36 arranged at predetermined intervals along the axial direction of the flow path 10. The heat generating plate 36 is made of a non-metal (e.g., silicon carbide) and generates heat when supplied with electricity. A plurality of through holes are formed at predetermined intervals in the heat generating plate 36, and the gas to be purified and ozone pass through the through holes. Each of the heat generating plates 36 supports a catalyst on its surface.

Also in this modification, the temperature of the catalyst increases as the gas to be purified and ozone, heated by the heat generating plate 36, come into contact with the catalyst, thereby promoting the reaction between methane and ozone on the catalyst. In addition, since the heat generating plate 36 is made of a non-metallic material, it is possible to suppress a decrease in ozone caused by a reaction with metal.

It should be noted that the methane purification apparatus 1 purifies methane contained in the air in the above description, but the present disclosure is not limited thereto. For example, the methane purification apparatus 1 may purify methane contained in exhaust gas discharged from an internal combustion engine of a vehicle or the like. In such a case, the methane purification apparatus 1 is provided in an exhaust passage of the internal combustion engine, and purifies methane in the exhaust gas flowing through the exhaust passage.

### <Effects of the embodiment>

The methane purification apparatus 1 of the above-described embodiment includes a non-metallic catalytic heater 32 that is provided downstream of the ozone supply unit 20 in the flow path 10 through which the gas to be purified containing methane flows and heats the gas to be purified and ozone. The catalytic heater 32 supports a catalyst that promotes the reaction between methane and ozone to purify methane.

When the catalytic heater 32 heats the gas to be purified and ozone, the temperature of the catalyst in the catalytic heater 32 increases. This promotes the reaction of methane and ozone on the catalyst. In addition, since the catalytic heater 32 is made of a non-metallic material, a decrease in ozone caused by a reaction with metal can be suppressed.

The present disclosure is explained on the basis of the exemplary embodiments. The technical scope of the present disclosure is not limited to the scope explained in the above embodiments and it is possible to make various changes and modifications within the scope of the disclosure. For example, all or part of the apparatus can be configured with any unit which is functionally or physically dispersed or integrated. Further, new exemplary embodiments generated by arbitrary combinations of them are included in the exemplary embodiments of the present disclosure. Further, effects of the new exemplary embodiments brought by the combinations also have the effects of the original exemplary embodiments.

1. A methane purification apparatus comprising:
a flow path through which a gas containing methane flows;
an ozone supply unit that supplies ozone to the gas; and
a non-metallic heater that is provided downstream of the ozone supply unit in the flow path and heats the gas and the ozone, wherein
the heater supports a catalyst for purifying the methane by causing the ozone to react with the methane.

2. The methane purification apparatus according to claim 1, wherein
the heater includes a heating element having a honeycomb structure, and
the heating element supports the catalyst on its surface.

3. The methane purification apparatus according to claim 2, wherein
the heating element is made of silicon carbide.

4. The methane purification apparatus according to claim 1, wherein
the heater includes a cylindrical carrier that supports the catalyst, and a heat generating unit that generates heat when electricity is supplied to the inside of the carrier.

5. The methane purification apparatus according to claim 1, wherein
the heater includes a plurality of flat plate-like heat generating plates arranged at predetermined intervals along an axial direction of the flow path, and
each of the plurality of heat generating plates supports the catalyst on its surface.

6. The methane purification apparatus according to claim 1, wherein
the catalyst includes any of a zeolite, an iron ion-exchanged zeolite, and a cobalt ion-exchanged zeolite.

7. The methane purification apparatus according to claim 1, further comprising:
a detection unit that detects temperature of the catalyst; and
a temperature control unit that operates the heater so that the temperature of the catalyst is below a first temperature at which the ozone decomposes.

8. The methane purification apparatus according to claim 7, wherein
the temperature control unit operates the heater so that the temperature of the catalyst is below the first temperature and at or above a second temperature at which moisture evaporates.

9. The methane purification apparatus according to claim 7, wherein
the temperature control unit alternately repeats a low-temperature control and a high-temperature control, the low-temperature control is performed by operating the heater so that the temperature of the catalyst is maintained at a third temperature, which is lower than the second temperature at which moisture evaporates, for a first time period and the high-temperature control is performed by operating the heater so that the temperature of the catalyst is maintained between the second temperature and the first temperature for a second time period, which is shorter than the first time period.
